(19) United States
(12) Patent Application Publication
(10) Pub. No.: US 2020/0369616 A1
(43) Pub. Date: Nov. 26, 2020

(54) ALKOXYCARBONATE ESTER PRODRUGS FOR USE AS ANTIMALARIAL AGENTS

(71) Applicants: OREGON HEALTH & SCIENCE UNIVERSITY, Portland, OR (US); THE UNITED STATES GOVERNMENT AS REPRESENTED BY THE DEPARTMENT OF VETERANS AFFAIRS, Washington, DC (US)

(72) Inventors: Michael K. Riscoe, Portland, OR (US); Sovitj Pou, Portland, OR (US); Rolf W. Winter, Portland, OR (US); Aaron C. Nilsen, Portland, OR (US); Stone S. Doggett, Portland, OR (US); Alina Kollenbrock, Portland, OR (US); Martin Smilkstein, Portland, OR (US); Yuexin Li, Portland, OR (US); Jane Kelly, Portland, OR (US); Rozalia Dodean, Portland, OR (US)

(21) Appl. No.: 16/883,974

(22) Filed: May 26, 2020

Related U.S. Application Data
(60) Provisional application No. 62/852,834, filed on May 24, 2019.

(51) Int. Cl. C07D 215/22; A61P 33/06

(52) U.S. Cl. CPC C07D 215/22; A61K 9/0019; A61P 33/06

(57) ABSTRACT

Provided are compounds of Formula (I):

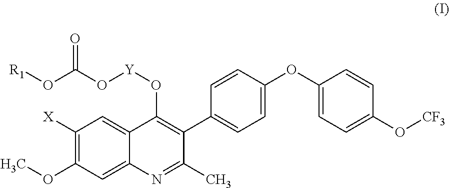

wherein: X is selected from the group of F and Cl; Y is selected from the group of —CH₂—, —CH₂—CH₂—, and —CH(CH₃)—; R₁ is selected from cycloalkyl, heterocyclyl, aromatic, heteroaromatic, and linear and branched alkyl, alkenyl, and alkynyl chains.

### GOVERNMENT SUPPORT

This invention was made with government support under A1100569 awarded by the National Institutes of Health and W81XWH-14-1-0447 awarded by the United States Army Medical Research and Materiel Command. The government has certain rights in the invention.

### FIELD OF THE INVENTION

This invention concerns novel alkoxycarbonate ester prodrug forms of antimalarial agents. More particularly, it concerns alkoxycarbonate ester prodrug forms of quinoline antimalarial agents.

### BACKGROUND OF THE INVENTION

Human malarial infection is initiated by the bite of an infected female Anopheles spp. mosquito. Sporozoites pour into the circulatory system from the salivary glands when the mosquito takes a blood meal and they quickly traffic to the liver where they invade and replicate. Within hepatocytes the parasites transform into merozoites and replicate to produce roughly 30,000 progeny per cell over the course of about 5 or 6 days. After this period the merozoites are released into the circulation where they invade red blood cells to begin a period of intraerythrocytic replication to expand the infectious burden roughly 20-fold per 48-hour cycle (for Plasmodium falciparum and P. vivax). After each round of this cycle the host red cell ruptures, thereby releasing its cargo of merozoites which initiate another round of red cell invasion and parasite expansion, leading to the legendary cyclical clinical symptoms in the host, i.e., fevers, chills, headaches, nausea, and vomiting. A small number of blood-stage merozoites exit the asexual replicative cycle to initiate differentiation into male and female gametocytes that are picked up by mosquitoes during the feeding process. Once inside the mosquito the gametocytes initiate the “sporogonic cycle” to form zygotes, ookinetes, oocysts, and ultimately sporozoites that migrate to the salivary glands to prime the mosquito vector for further transmission of the disease.

Malaria remains one of the deadliest diseases in the world today, as it has been for thousands of years. For each of the ≈0.4 million people killed each year, hundreds of millions more suffer from severe illness (Sachs J, Malaney P. 2002. The economic and social burden of malaria. Nature 415:680-685). Spread by mosquitoes from person to person malaria remains one of the most widespread infectious diseases of our time. There are six identified species of the parasite responsible for human malaria all belonging to genus Plasmodium. P. falciparum is the dominant species in sub-Saharan Africa, and is responsible for the majority of malaria-related deaths. P. vivax, responsible for relapsing malaria, causes as much as 25-40% of the global malaria burden, whereas P. ovale, and P. malariae represent a small percentage of infections. It is now recognized that a 5th and 6t^{h} species of subhuman primates, P. knowlesi and P. cynomolgi, are a significant cause of malaria in parts of SE Asia, particularly Malaysia (Barber et al., 2017. World Malaria Report: time to acknowledge Plasmodium knowlesi malaria. Malar J 16:135; Imwong et al., 2018. Asymptomatic Natural Human Infections With the Simian Malaria Parasites Plasmodium cynomolgi and Plasmodium knowlesi. J Infect Dis doi:10.1093/infdis/jiy519.; and Oddux et al., Identification of the five human Plasmodium species including P. knowlesi by real-time polymerase chain reaction. Eur J Clin Microbiol Infect Dis 30:597-601).

Major advances have been made in the fight against malaria over the past 2 decades. Through the use of insecticide-treated bed nets, vector control measures, and the introduction of artemisinin combination therapies (ACTs), deaths from malaria have fallen by over 60% since the year 2000 and 17 countries have eliminated the disease in the last decade. But over 400,000 people, mostly children, still die from malaria each year, and this does not include the tens of thousands added annually who survive malaria but suffer persistent neuro-cognitive consequences after cerebral malaria or deficits in growth, health, and brain function from being born to a mother with malaria during pregnancy. Re-examination of existing strategies to reduce these sequelae, and to ultimately eradicate the disease, reveals fundamental reasons why novel pharmacologic approaches are still urgently needed: (1. gains made in controlling the disease are now threatened by the emergence of artemisinin resistance in SE Asia (Ashley et al., 2014. Spread of artemisinin resistance in Plasmodium falciparum malaria. N Engl J Med 371:411-423; and Dondorp et al., 2009. Artemisinin resistance in Plasmodium falciparum malaria. N Engl J Med 361:455-467), (2. sustained chemo-prophylaxis is essential for both disease prevention and eradication strategies (Burrows et al., 2017. New developments in anti-malarial target candidate and product profiles. Malar J 16:26), and (3. the discovery, development, and licensing of a malaria vaccine formulation, which meets all necessary regulatory requirements, including safety, affordability, accessibility, applicability, and sterile protective efficacy is far beyond the horizon (Matuschewski K. 2017. Vaccines against malaria-still a long way to go. FEBS J 284:2560-2568). The parasite mitochondrion is targeted by atovaquone (ATV) which inhibits the parasite cyt bc₁ complex (Hudson AT. 1993. Atovaquone—a novel broad-spectrum anti-infective drug. Parasitol Today 9:66-68). ATV (FIG. 1) is combined with proguanil because monotherapy leads to drug resistance (Looareesuwan et al., 1996. Clinical studies of atovaquone, alone or in combination with other antimalarial drugs, for treatment of acute uncomplicated malaria in Thailand. Am J Trop Med Hyg 54:62-66).

ELQ-300 is a novel antimalarial drug that, like atovaquone, targets the P. falciparum cyt bc₁ complex and can be used for treatment of and prophylaxis against malaria as well as for transmission blocking (Nilsen et al., 2013. Quinolone-3-diarylethers: a new class of antimalarial drug. Sci Transl Med 5:177ra137). Chemical synthesis of ELQ-300 was described by us in 2014 (Nilsen et al., 2014. Discovery, Synthesis, and Optimization of Antimalarial 4(1H)-Quinolone-3-Diarylethers. J Med Chem 57:3818-3834). The drug has many positive attributes including:

1) low nanomolar IC₅₀'s vs. multidrug resistant P. falciparum strains (Nilsen et al., Sci Transl Med 5:177ra137),

2) equal potency vs. ATV resistant parasites ((Nilsen et al., Sci Transl Med 5:177ra137; and Nilsen et al., J Med Chem 57:3818-3834),

3) 30-fold more effective than ATV in vivo in treatment of malaria-infected mice (ED₅₀=0.02 mg/kg and curative at 0.3 to 1 mg/kg in 4-day Peters Test) ((Nilsen et al., Sci Transl Med 5:177ra137),

4) metabolically stable in presence of mouse, rat, and human microsomes ((Nilsen et al., Sci Transl Med 5:177ra137),

5) very low propensity for resistance (i.e., less than 1 in 10⁹) relative to ATV ((Nilsen et al., Sci Transl Med 5:177ra137),

6) novel site of action—targets the highly divergent Qᵢ site of P. falciparum cyt bc₁ (ATV targets the distant Qₒ site) which in turn blocks the coQ cycle leading to pyrimidine starvation and killing of the parasite (13, 14),

7) targets actively replicating parasites in the liver, bloodstream, and vector stages of infection ((Nilsen et al., Sci Transl Med 5:177ra137),

8) allometric scaling predicts a very long t_{1/2} in humans ≈120 hours ((Nilsen et al., Sci Transl Med 5:177ra137),

9) cost effective chemical synthesis procedures have been developed (Nilsen et al., J Med Chem 57:3818-3834, 2014), and

10) high selectivity, 10,000-fold, for the parasite over the host cyt bc₁ ((Nilsen et al., Sci Transl Med 5:177ra137).

ELQ-331 is a bioreversible prodrug that yields ELQ-300 upon esterase cleavage (Frueh et al., 2017. Alkoxycarbonate Ester Prodrugs of Preclinical Drug Candidate ELQ-300 for Prophylaxis and Treatment of Malaria. ACS Infect Dis 3:728-735). Because of reduced crystallinity compared to ELQ-300 it is possible to formulate ELQ-331 to deliver single dose cures of patent malaria infections in mice by oral drug delivery (Frueh et al, ACS Infect Dis 3:728-735). Similar but somewhat less impressive results were observed for the ethylcarbonate prodrug, ELQ-337, as described by Miley et al. (2015. ELQ-300 Prodrugs for Enhanced Delivery and Single-Dose Cure of Malaria. Antimicrob Agents Chemother 59:5555-5560).

There remains a need for new prodrug forms of antimalarial compounds for animal and human use.

### BRIEF SUMMARY OF THE INVENTION

Provided are compounds of Formula (I):

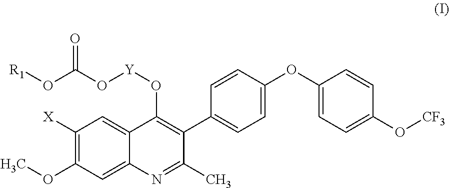

### Wherein:

X is selected from the group of F and Cl;

Y is selected from the group of —CH₂—, —CH₂—CH₂—, and —CH(CH₃)—;

R₁ is selected from the group of:
a) —CH₃;
b) n-propyl;
c) C₄-C₂₀ linear or branched alkyl;
d) —(C₄-C₂₀ branched or linear alkylene)-C₃-C₆ cycloalkyl;
e) —(C₄-C₂₀ branched or linear alkylene)-C₃-C₆ heterocyclyl;
f) —(C₄-C₂₀ branched or linear alkylene)-C₃-C₆ cycloalkene;
g) —(C₄-C₂₀ branched or linear alkylene)-phenyl;
h) —(C₄-C₂₀ branched or linear alkylene)-naphthyl;
i) C₃-C₆ cycloalkyl;
j) C₃-C₆ heterocyclyl;
k) C₃-C₆ cycloalkene;
l) phenyl;
m) naphthyl;
n) C₂-C₂₀ linear or branched alkyl;
o) —(C₂-C₂₀ linear or branched alkenylene)-C₃-C₆ cycloalkyl;
p) —(C₂-C₂₀ linear or branched alkenylene)-C₃-C₆ heterocycle;
q) —(C₂-C₂₀ linear or branched alkenylene)-C₃-C₆ cycloalkene;
r) —(C₄-C₂₀ branched or linear alkenylene)-phenyl;
s) —(C₄-C₂₀ branched or linear alkenylene)-naphthyl;
t) C₂-C₂₀ linear or branched alkyne;
u) —(C₂-C₂₀ linear or branched alkynylene)-C₃-C₆ cycloalkyl;
v) —(C₂-C₂₀ linear or branched alkynylene)-C₃-C₆ heterocycle;
w) —(C₂-C₂₀ linear or branched alkynylene)-C₃-C₆ cycloalkene;
x) —(C₄-C₂₀ branched or linear alkynylene)-phenyl;
y) —(C₄-C₂₀ branched or linear alkynylene)-naphthyl;
   and
z) —(CH₂—CH₂—O—)₁₋₅R₂;

and

R₂ is selected from the group of methyl and ethyl;

or a pharmaceutically acceptable salt thereof.

Also disclosed herein are compositions comprising a pharmaceutically or therapeutically active amount of at least one compound of Formula I, or a pharmaceutically acceptable salt thereof, and at least one pharmaceutically acceptable carrier.

Further disclosed herein are methods for inhibiting a parasitic or infectious disease in a subject comprising administering to the subject a therapeutically effective amount of a compound of Formula I, or a pharmaceutically acceptable salt thereof.

### BRIEF DESCRIPTION OF THE DRAWINGS

FIG. 1 charts ELQ-331 and ELQ-300 pharmacokinetics after IM ELQ-331 injection and Plasma ELQ-300 concentration after ELQ-331 injection.

FIG. 2 depicts bioluminescent imaging of the protective effect of IM ELQ-300 prodrugs on Plasmodium yoelii liver stages.

### DETAILED DESCRIPTION OF THE INVENTION

An embodiment provides a compound of Formula (I), above, or a pharmaceutically acceptable salt thereof, wherein Y is —CH₂— and each of X, R₁, and R2 are as defined above. A separate embodiment provides a compound of Formula (I), above, or a pharmaceutically acceptable salt thereof, wherein Y is —CH₂—, X is Cl, and each of R₁, and R₂ are as defined above. Another separate embodiment provides a compound of Formula (I), above, or a pharmaceutically acceptable salt thereof, wherein Y is —CH₂—, X is F, and each of R₁, and R2 are as defined above.

Another embodiment provides a compound of Formula (I), above, or a pharmaceutically acceptable salt thereof, wherein Y is —CH₂—CH₂— and each of X, R₁, and R₂ are as defined above. A separate embodiment provides a compound of Formula (I), above, or a pharmaceutically acceptable salt thereof, wherein Y is —CH₂—CH₂—, X is Cl, and each of R₁, and R₂ are as defined above. Another separate embodiment provides a compound of Formula (I), above, or a pharmaceutically acceptable salt thereof, wherein Y is —CH₂—CH₂—, X is F, and each of R₁, and R₂ are as defined above.

A further embodiment provides a compound of Formula (I), above, or a pharmaceutically acceptable salt thereof, wherein Y is —CH(CH₃)— and each of X, R₁, and R₂ are as defined above. A separate embodiment provides a compound of Formula (I), above, or a pharmaceutically acceptable salt thereof, wherein Y is —CH(CH₃)—, X is Cl, and each of R₁, and R₂ are as defined above. Another separate embodiment provides a compound of Formula (I), above, or a pharmaceutically acceptable salt thereof, wherein Y is —CH(CH₃)—, X is F, and each of R₁, and R₂ are as defined above.

Also provided are compounds of Formula (I):

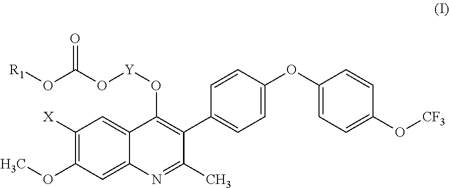

wherein:

X is selected from the group of F and Cl;

Y is selected from the group of —CH₂—, —CH₂—CH₂—, and —CH₂(CH₃)—;

R₁ is selected from the group of:

a) C₄-C₂₀ branched or linear alkyl;
b) —(CH₂—CH₂—O—)₂₋₅—R₂;
c) C₂-C₂₀ linear or branched alkenyl;

and

R₂ is selected from the group of methyl and ethyl;

or a pharmaceutically acceptable salt thereof.

Also provided are compounds of Formula (II):

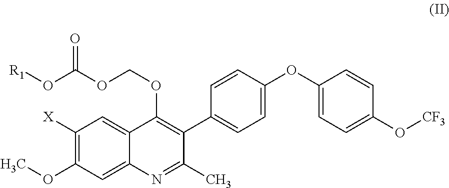

wherein:

X is selected from the group of F and Cl;

R₁ is selected from the group of:
a) C₄-C₂₀ branched or linear alkyl;
b) —(CH₂—CH₂—O—)₂₋₅—R₂;
c) C₂-C₂₀ linear or branched alkenyl;

and

R₂ is selected from the group of methyl and ethyl;

or a pharmaceutically acceptable salt thereof.

Further embodiments provide a compound of Formula (I) and Formula (II), respectively, in which X is F, and all other variables are as previously described.

Other embodiments provide a compound of Formula (I) and Formula (II), respectively, in which X is Cl, and all other variables are as previously described.

Within each of the embodiments described herein, there is a further embodiment in which R₁ is C₄-C₁₀ branched or linear alkylene, and all other variables are as described for the embodiment referenced.

Within each of the embodiments described herein, there is a further embodiment in which R₁ is C₆-C₁₀ branched or linear alkylene, and all other variables are as described for the embodiment referenced.

Within each of the embodiments described herein, there is a further embodiment in which R₁ is C₈-C₁₀ branched or linear alkylene, and all other variables are as described for the embodiment referenced.

Within each of the embodiments described herein, there is a further embodiment in which R₁ is C₄-C₂₀ branched or linear alkylene, and all other variables are as described for the embodiment referenced.

Within each of the embodiments described herein, there is a further embodiment in which R₁ is C₆-C₂₀ branched or linear alkylene, and all other variables are as described for the embodiment referenced.

Within each of the embodiments described herein, there is a further embodiment in which R₁ is C₈-C₂₀ branched or linear alkylene, and all other variables are as described for the embodiment referenced.

Within each of the embodiments described herein, there is a further embodiment in which R₁ is C₁₀-C₂₀ branched or linear alkylene, and all other variables are as described for the embodiment referenced.

Within each of the embodiments described herein, there is a further embodiment in which R₁ is C₁₂-C₂₀ branched or linear alkylene, and all other variables are as described for the embodiment referenced.

Within each of the embodiments described herein, there is a further embodiment in which R₁ is C₄-C₁₆ branched or linear alkylene, and all other variables are as described for the embodiment referenced.

Within each of the embodiments described herein, there is a further embodiment in which R₁ is C₆-C₁₆ branched or linear alkylene, and all other variables are as described for the embodiment referenced.

Within each of the embodiments described herein, there is a further embodiment in which R₁ is C₈-C₁₆ branched or linear alkylene, and all other variables are as described for the embodiment referenced.

Within each of the embodiments described herein, there is a further embodiment in which R₁ is C₁₀-C₁₆ branched or linear alkylene, and all other variables are as described for the embodiment referenced.

Within each of the embodiments described herein, there is a further embodiment in which R₁ is C₄-C₁₄ branched or linear alkylene, and all other variables are as described for the embodiment referenced.

Within each of the embodiments described herein, there is a further embodiment in which R₁ is C₆-C₁₄ branched or linear alkylene, and all other variables are as described for the embodiment referenced.

Within each of the embodiments described herein, there is a further embodiment in which R₁ is C₈-C₁₄ branched or linear alkylene, and all other variables are as described for the embodiment referenced.

Within each of the embodiments described herein, there is a further embodiment in which R₁ is C₁₀-C₁₄ branched or linear alkylene, and all other variables are as described for the embodiment referenced.

Within each of the embodiments described herein, there is a further embodiment in which R₁ is C₄-C₁₂ branched or linear alkylene, and all other variables are as described for the embodiment referenced.

Within each of the embodiments described herein, there is a further embodiment in which R₁ is C₆-C₁₂ branched or linear alkylene, and all other variables are as described for the embodiment referenced.

Within each of the embodiments described herein, there is a further embodiment in which R₁ is C₈-C₁₂ branched or linear alkylene, and all other variables are as described for the embodiment referenced.

Within each of the embodiments described herein, there is a further embodiment in which R₁ is C₁₀-C₁₂ branched or linear alkylene, and all other variables are as described for the embodiment referenced.

Within each of the embodiments described herein, there is a further embodiment in which R₁ is C₂-C₂₀ branched or linear alkenylene, and all other variables are as described for the embodiment referenced.

Within each of the embodiments described herein, there is a further embodiment in which R₁ is C₄-C₂₀ branched or linear alkenylene, and all other variables are as described for the embodiment referenced.

Within each of the embodiments described herein, there is a further embodiment in which R₁ is C₆-C₂₀ branched or linear alkenylene, and all other variables are as described for the embodiment referenced.

Within each of the embodiments described herein, there is a further embodiment in which R₁ is C₈-C₂₀ branched or linear alkenylene, and all other variables are as described for the embodiment referenced.

Within each of the embodiments described herein, there is a further embodiment in which R₁ is C₁₀-C₂₀ branched or linear alkenylene, and all other variables are as described for the embodiment referenced.

Within each of the embodiments described herein, there is a further embodiment in which R₁ is C₁₂-C₂₀ branched or linear alkenylene, and all other variables are as described for the embodiment referenced.

Within each of the embodiments described herein, there is a further embodiment in which R₁ is C₄-C₁₆ branched or linear alkenylene, and all other variables are as described for the embodiment referenced.

Within each of the embodiments described herein, there is a further embodiment in which R₁ is C₆-C₁₆ branched or linear alkenylene, and all other variables are as described for the embodiment referenced.

Within each of the embodiments described herein, there is a further embodiment in which R₁ is C₈-C₁₆ branched or linear alkenylene, and all other variables are as described for the embodiment referenced.

Within each of the embodiments described herein, there is a further embodiment in which R₁ is C₁₀-C₁₆ branched or linear alkenylene, and all other variables are as described for the embodiment referenced.

Within each of the embodiments described herein, there is a further embodiment in which R₁ is C₄-C₁₄ branched or linear alkenylene, and all other variables are as described for the embodiment referenced.

Within each of the embodiments described herein, there is a further embodiment in which R₁ is C₆-C₁₄ branched or linear alkenylene, and all other variables are as described for the embodiment referenced.

Within each of the embodiments described herein, there is a further embodiment in which R₁ is C₈-C₁₄ branched or linear alkenylene, and all other variables are as described for the embodiment referenced.

Within each of the embodiments described herein, there is a further embodiment in which R₁ is C₁₀-C₁₄ branched or linear alkenylene, and all other variables are as described for the embodiment referenced.

Within each of the embodiments described herein, there is a further embodiment in which R₁ is C₄-C₁₂ branched or linear alkenylene, and all other variables are as described for the embodiment referenced.

Within each of the embodiments described herein, there is a further embodiment in which R₁ is C₆-C₁₂ branched or linear alkenylene, and all other variables are as described for the embodiment referenced.

Within each of the embodiments described herein, there is a further embodiment in which R₁ is C₈-C₁₂ branched or linear alkenylene, and all other variables are as described for the embodiment referenced.

Within each of the embodiments described herein, there is a further embodiment in which R₁ is C₁₀-C₁₂ branched or linear alkenylene, and all other variables are as described for the embodiment referenced.

Within each of the embodiments described herein, there is a further embodiment in which R₁ is —(CH₂—CH₂—O—)₁₋₅—R₂ and R₂ is methyl or ethyl.

Within each of the embodiments described herein, there is a further embodiment in which R₁ is —(CH₂—CH₂—O—)₂₋₅—R₂ and R₂ is methyl or ethyl.

Within each of the embodiments described herein, there is also a further embodiment in which R₁ is —(CH₂—CH₂—O—)₃₋₅—R₂ and R₂ is methyl or ethyl.

Within each of the embodiments described herein, there is also a further embodiment in which R₁ is —(CH₂—CH₂—O—)₄₋₅—R₂ and R₂ is methyl or ethyl.

For the definitions of R₁ herein, a branched or linear, or cyclic alkyl group is understood to be a chain of a specified number of carbon atoms (for example, 4 to 20 carbon atoms) that may also be branched by additional alkyl, alkenyl, or alkynyl groups, each of which may be further substituted by branching alkyl, alkenyl, alkynyl, cycloalkyl, heterocycle, cycloalkane, phenyl, or naphthyl groups, that are not counted in the number of the longest chain. For instance, each of the groups below, in which the wavy line represents the bond from which the alkyl group extends, would be considered a 9-carbon or C₉-branched alkyl group or chain, wherein the bolded lines depict the longest chain from which other groups branch.

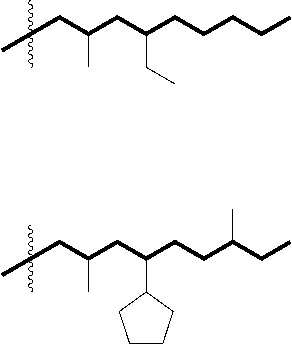

Similarly, R₁ groups defined as linear or branched alkenyl refer to groups having from 2 to 20 carbon atoms in the longest designated chain, though additional carbon/alkyl/alkylene substituents may branch from the alkylene chain. For instance, both groups below would be C₉-alkylene chains under R₁.

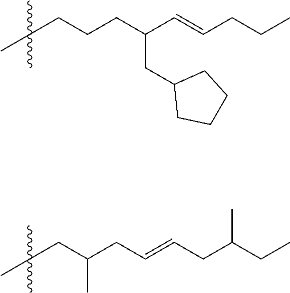 [SMILES: *CCCC(/C=C/CCC)CC1CCCC1 ; *CC(C)C/C=C/CC(C)CC]

In some embodiments herein, the cited R₁ chains listed as C₄-C₂₀ branched or linear alkylene, C₄-C₂₀ branched or linear alkenylene, and C₄-C₂₀ branched or linear alkynylene, as well as the shorter cited chains of each, are linear chains. In other embodiments, each of the cited branched alkylene, branched alkenylene, and branched alkynylene chains is substituted by 1, 2, 3, 4 or 5 substituents selected from the group of C₁-C₆ alkyl, C₂-C₆ alkenyl, C₂-C₆ alkynyl, C₃-C₆ cycloalkyl, —(C₁-C₃ alkylene)-C₃-C₆ cycloalkyl, 3-6 membered heterocyclyl groups, and —(C₁-C₃ alkylene)-3-6 membered heterocyclyl groups.

In other embodiments, each of the cited branched alkylene, branched alkenylene, and branched alkynylene chains is substituted by 1, 2, 3, 4 or 5 substituents selected from the group of C₁-C₆ alkyl, C₂-C₆ alkenyl, C₂-C₆ alkynyl, C₃-C₆ cycloalkyl, and —(C₁-C₃ alkylene)-C₃-C₆ cycloalkyl groups.

In further embodiments, each of the cited branched alkylene, branched alkenylene, and branched alkynylene chains is substituted by 1, 2, 3, 4 or 5 substituents selected from the group of C₁-C₄ alkyl, C₂-C₄ alkenyl, C₂-C₄ alkynyl, C₃-C₆ cycloalkyl, and —CH₂—C₃—C₆ cycloalkyl groups.

In still further embodiments, each of the cited branched alkylene, branched alkenylene, and branched alkynylene chains is substituted by 1, 2, 3, 4 or 5 C₁-C₆ alkyl substituents.

In still further embodiments, each of the cited branched alkylene, branched alkenylene, and branched alkynylene chains is substituted by 1, 2, 3, 4 or 5 C₁-C₄ alkyl substituents.

In still further embodiments, each of the cited branched alkylene, branched alkenylene, and branched alkynylene chains is substituted by 1, 2, 3, 4 or 5 C₁-C₃ alkyl substituents.

In each of the cited branched alkylene, branched alkenylene, and branched alkynylene chains discussed above, it is understood that the 1, 2, 3, 4 or 5 substituents comprise the “branching” portion(s) of the chain

Also provided herein is a pharmaceutical composition comprising a pharmaceutically effective amount of a compound of Formula (I), or a pharmaceutically acceptable salt thereof, and a pharmaceutically acceptable carrier or excipient.

Also provided herein is a pharmaceutical composition comprising a pharmaceutically effective amount of a compound of Formula (II), or a pharmaceutically acceptable salt thereof, and a pharmaceutically acceptable carrier or excipient.

Also provided is a method for inhibiting malaria or toxoplasmosis in a human in need thereof comprising administering to the human a therapeutically effective amount of a compound of Formula (I), or a pharmaceutically acceptable salt thereof.

Also provided is a method for inhibiting malaria or toxoplasmosis in a human in need thereof comprising administering to the human a therapeutically effective amount of a compound of Formula (II), or a pharmaceutically acceptable salt thereof.

Also provided is a method for inhibiting babesiosis in a human in need thereof comprising administering to the human a therapeutically effective amount of a compound of Formula (I), or a pharmaceutically acceptable salt thereof.

Also provided is a method for inhibiting babesiosis in a human in need thereof comprising administering to the human a therapeutically effective amount of a compound of Formula (II), or a pharmaceutically acceptable salt thereof.

Also provided is a method for inhibiting toxoplasmosis in a human in need thereof comprising administering to the human a therapeutically effective amount of a compound of Formula (I), or a pharmaceutically acceptable salt thereof.

Also provided is a method for inhibiting toxoplasmosis in a human in need thereof comprising administering to the human a therapeutically effective amount of a compound of Formula (II), or a pharmaceutically acceptable salt thereof.

For each method for inhibiting malaria described herein, there is an embodiment wherein the malaria is multidrug-resistant malaria. For each method for inhibiting malaria described herein, there is an embodiment wherein the malaria is chloroquine-resistant malaria.

For each method for inhibiting malaria described herein, there is an embodiment wherein the compound of Formula (I) or Formula (II), or a pharmaceutically acceptable salt thereof, is co-administered to the human in need thereof with at least one additional anti-malarial agent or drug.

### Definitions

The modifier “about” used in connection with a quantity is inclusive of the stated value and has the meaning dictated by the context (e.g., includes the degree of error associated with measurement of the particular quantity). In some embodiments the term “about” refers to the amount indicated, plus or minus 10%. In some embodiments the term “about” refers to the amount indicated, plus or minus 5%.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

“Inhibiting” (which is inclusive of “treating”) refers to inhibiting the full development of a disease or condition, for example, in a subject who is at risk for a disease such as malaria. “Treatment” refers to a therapeutic intervention that ameliorates a sign or symptom of a disease or pathological condition after it has begun to develop. As used herein, the term “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. “Inhibiting” also refers to any quantitative or qualitative reduction including prevention of infection or complete killing of an invading organism, relative to a control. A “prophylactic” treatment is a treatment administered to a subject who does not exhibit signs of a disease or exhibits only early signs for the purpose of decreasing the risk of developing pathology. By the term “coadminister” is meant that each of at least two compounds be administered during a time frame wherein the respective periods of biological activity overlap. Thus, the term includes sequential as well as coextensive administration of two or more drug compounds.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the number of carbon atoms in a linear alkyl, alkene, or alkyne chain in the range of “from 2 to 20” in C₂-C₂₀ is inclusive of the endpoints, 2 and 20, and all the intermediate values).

The term “alkyl” refers to a straight or branched hydrocarbon. For example, an alkyl group can include those having 4 to 20 carbon atoms (i.e, C₄-C₂₀ alkyl), 4 to 12 carbon atoms (i.e., C₄-C₁₂ alkyl), or 8 to 12 carbon atoms (i.e., C₈-C₁₂ alkyl). Examples of suitable alkyl groups include, but are not limited to, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, octyl (—(CH₂)₇CH₃), nonyl (—(CH₂)₈CH₃), decyl (—(CH₂)₉CH₃), undecyl (—(CH₂)₁₀CH₃), dodecyl (—(CH₂)₁₁CH₃), tridecyl (—(CH₂)₁₂CH₃), tetradecyl (—(CH₂)₁₃CH₃), pentadecyl (—(CH₂)₁₄CH₃), hexadecyl (—(CH₂)₁₅CH₃), octadecyl (—(CH₂)₁₇CH₃), and eicosyl (—(CH₂)₁₉CH₃).

The term “cycloalkyl” refers to a saturated ring having 3 to 6 carbon atoms as a monocycle, including cyclopropyl, cyclobutyl, cyclopentyl, and cyclohexyl groups.

The term “heterocycle” or “heterocyclyl” as used herein includes by way of example and not limitation those heterocycles described in Paquette, Leo A.; Principles of Modern Heterocyclic Chemistry (W. A. Benjamin, New York, 1968), particularly Chapters 1, 3, 4, 6, 7, and 9; The Chemistry of Heterocyclic Compounds, A Series of Monographs” (John Wiley & Sons, New York, 1950 to present), in particular Volumes 13, 14, 16, 19, and 28; and J. Am. Chem. Soc. (1960) 82:5566. The term “heterocyclyl” may be understood as a monovalent heterocyclic ring bound to a carbon chain or other group. In one specific embodiment of the invention “heterocycle” includes a “carbocycle” as defined herein, wherein one or more (e.g. 1, 2, 3, or 4) carbon atoms have been replaced with a heteroatom (e.g. O, N, or S). The terms “heterocycle” or “heterocyclyl” includes saturated rings (such as tetrahydrofuranyl, pyrrolidinyl, piperidinyl and morpholinyl groups), partially unsaturated rings, and aromatic rings (i.e., heteroaromatic rings, such as furanyl, thiophenyl, pyrrolyl, imidazolyl, pyridinyl, and pyrimidinyl groups). For each embodiment herein comprising a group of any selected from heterocyclyl, —(C₄-C₂₀ branched or linear alkyl)-C₃-C₆ heterocyclyl, —(C₂-C₂₀ linear or branched alkenyl)-C₃-C₆ heterocycle, and —(C₂-C₂₀ linear or branched alkynyl)-C₃-C₆ heterocyclyl, there is a further embodiment in which the heterocyclyl ring is a fully saturated heterocyclyl ring. In another further embodiment, the heterocyclyl ring of any of the heterocyclyl, —(C₄-C₂₀ branched or linear alkyl)-C₃-C₆ heterocyclyl, —(C₂-C₂₀ linear or branched alkenyl)-C₃-C₆ heterocycle, and —(C₂-C₂₀ linear or branched alkynyl)-C₃-C₆ heterocyclyl groups is a partially unsaturated heterocyclyl ring. In still another embodiment in each case, the heterocyclyl ring is an aromatic heterocyclyl ring.

The terms “cycloalkenyl” or “cycloalkene” refer to a partially unsaturated ring having from 3 to 6 carbon atoms as a monocycle, including as non-limiting examples 1-cyclopent-1-yl, 2-cyclopent-1-yl, 3-cyclopent-1-yl, 1-cyclohexen-1-yl, 2-cyclohexen-1-yl, and 3-cyclohexen-1-yl groups.

The term “alkenyl” refers to a straight or branched hydrocarbon with at least one site of unsaturation, i.e. a carbon-carbon, sp² double bond. For example, an alkenyl group can have 2 to 20 carbon atoms (i.e., C₂-C₂₀ alkenyl), 2 to 8 carbon atoms (i.e., C₂-C₈ alkenyl), or 2 to 6 carbon atoms (i.e., C₂-C₆ alkenyl). Examples of suitable alkenyl groups include, but are not limited to, ethylene or vinyl (—CH═CH₂), allyl (—CH2CH═CH₂), cyclopentenyl (—O₅H₇), and 5-hexenyl (—CH₂CH₂CH₂CH₂CH═CH₂). The term “alkynyl” refers to a straight or branched hydrocarbon with at least one site of unsaturation, i.e. a carbon-carbon, sp triple bond. For example, an alkynyl group can have 2 to 20 carbon atoms (i.e., C2-C20 alkynyl), 2 to 8 carbon atoms (i.e., C₂-C₈ alkyne), or 2 to 6 carbon atoms (i.e., C₂-C₆ alkynyl). Examples of suitable alkynyl groups include, but are not limited to, acetylenic (—CΞCH), propargyl (—CH₂CΞCH), and the like.

The term “alkylene” refers to a bivalent saturated aliphatic radical (such as ethylene or propylene) prepared from an alkane by removal of two hydrogen atoms from different carbon atoms.

The term “alkenylene” refers to a bivalent saturated aliphatic radical prepared from an alkene (such as propenylene) by removal of two hydrogen atoms from different carbon atoms.

The term “alkynylene” refers to a bivalent saturated aliphatic radical prepared from an alkyne by removal of two hydrogen atoms from different carbon atoms.

“Multidrug-resistant” or “drug-resistant” refers to malaria, or the parasites causing malaria, that have developed resistance to treatment by at least one therapeutic agent historically administered to treat malaria. For example, there are multidrug-resistant strains of Plasmodium falciparum that harbor high-level resistance to chloroquine, quinine, mefloquine, pyrimethamine, sulfadoxine and atovaquone.

The terms “pharmaceutically acceptable salt(s)” or “pharmacologically acceptable salt(s)” refer to salts prepared by conventional means that include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. A “pharmaceutically acceptable salt” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977).

The term “pharmacologically active amount” relates to an amount of a compound that provides a detectable reduction in parasitic activity in vitro or in vivo, or diminishes the likelihood of emergence of drug resistance.

A “therapeutically effective amount” or “diagnostically effective amount” refers to a quantity of a specified agent sufficient to achieve a desired effect in a subject being treated with that agent. For example, this may be the amount of a compound disclosed herein useful in detecting or treating thyroid cancer in a subject. Ideally, a therapeutically effective amount or diagnostically effective amount of an agent is an amount sufficient to inhibit or treat the disease without causing a substantial cytotoxic effect in the subject. The therapeutically effective amount or diagnostically effective amount of an agent will be dependent on the subject being treated, the severity of the affliction, and the manner of administration of the therapeutic composition.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The suitability and techniques involved in making and using prodrugs are well known by those skilled in the art. For a general discussion of prodrugs involving esters see Svensson and Tunek Drug Metabolism Reviews 165 (1988) and Bundgaard Design of Prodrugs, Elsevier (1985).

The term “prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs include compounds having a phosphonate and/or amino group functionalized with any group that is cleaved in vivo to yield the corresponding amino and/or phosphonate group, respectively.

Particular examples of the presently disclosed compounds include one or more asymmetric centers; thus these compounds can exist in different stereoisomeric forms. Accordingly, compounds and compositions may be provided as individual pure enantiomers or as stereoisomeric mixtures, including racemic mixtures. In certain embodiments the compounds disclosed herein are synthesized in or are purified to be in substantially enantiopure form, such as in a 90% enantiomeric excess, a 95% enantiomeric excess, a 97% enantiomeric excess or even in greater than a 99% enantiomeric excess, such as in enantiopure form.

It is understood that substituents and substitution patterns of the compounds described herein can be selected by one of ordinary skill in the art to provide compounds that are chemically stable and that can be readily synthesized by techniques known in the art and further by the methods set forth in this disclosure. Reference will now be made in detail to the presently preferred compounds.

The compounds and pharmaceutical compositions disclosed herein can be used for inhibiting or preventing parasitic diseases. For example, human or animal parasitic diseases include malaria, toxoplasmosis, amebiasis, giardiasis, leishmaniasis, trypanosomiasis, and coccidiosis, caused by organisms such as Toxoplasma sp. (such as Toxoplasma gondii), Eimeria sp. (Eimeriosis), Babesia bovis (babesiosis), Theileria sp. (Theileria annulata⇒tropical theileriosis and Theileria parva—East Coast fever), and also includes infections by helminths, such as ascaris, schistosomes and filarial worms. The compounds and compositions are also effective in the inhibition of fungal pathogens including Pneumocystis carinii, Aspergillus fumigatus, and others.

In particular embodiments, the parasitic diseases may be caused by parasites that cause malaria. Particular species of parasites that are included within this group include all species that are capable of causing human or animal infection. Illustrative species include Plasmodium falciparum, Plasmodium vivax, Plasmodium ovale, Plasmodium knowlesi, and Plasmodium malariae. The compounds and compositions disclosed herein are particularly useful for inhibiting drug-resistant malaria such as chloroquine-resistant malaria or multidrug-resistant malaria that is caused by organisms harboring resistance to chloroquine, quinine, mefloquine, pyrimethamine, dapsone, and/or atovaquone.

Toxoplasmosis is caused by a sporozoan parasite of the Apicomplexa called Toxoplasma gondii. It a common tissue parasite of humans and animals. Most of the infections appear to be asymptomatic (90%), however toxoplasmosis poses a serious health risk for immuno-compromised individuals, such as organ transplant recipients, cancer and AIDS patients, and the unborn children of infected mothers. The compounds disclosed herein may be used alone to treat toxoplasmosis or they may be co-administered with “antifolates” such as sulfonamides, pyrimethamine, tirmethoprim, biguanides and/or atovaquone.

In further embodiments, the compounds disclosed herein may be co-administered with another pharmaceutically active compound. For example, the compounds may be co-administered with quinine, chloroquine, atovaquone, proguanil, primaquine, amodiaquine, mefloquine, piperaquine, artemisinin, methylene blue, pyrimethamine, sulfadoxine, artemether-lumefantrine (Coartem®), dapsone-chlorproguanil (LAPDAP®), artesunate, quinidine, clopidol, pyridine/pyridinol analogs, 4(1H)-quinolone analogs, dihydroartemisinin, a mixture of atovaquone and proguanil, an endoperoxide, an acridone as disclosed in WO 2008/064011 (which is incorporated herein by reference in its entirety), a pharmachin as disclosed in U.S. Provisional Patent Application titled “Compounds for Treating Parasitic Disease” filed Nov. 18, 2008 (which is incorporated herein by reference in its entirety), or any combination of these.

The compounds disclosed herein may be included in pharmaceutical compositions (including therapeutic and prophylactic formulations), typically combined together with one or more pharmaceutically acceptable vehicles or carriers and, optionally, other therapeutic ingredients (for example, antibiotics, anti-inflammatories, or drugs that are used to reduce pruritus such as an antihistamine). The compositions disclosed herein may be advantageously combined and/or used in combination with other antimalarial agents as described above.

Such pharmaceutical compositions can be administered to subjects by a variety of mucosal administration modes, including by oral, rectal, intranasal, intrapulmonary, or transdermal delivery, or by topical delivery to other surfaces. Optionally, the compositions can be administered by non-mucosal routes, including by intramuscular, subcutaneous, intravenous, intra-arterial, intra-articular, intraperitoneal, intrathecal, intracerebroventricular, or parenteral routes. In other alternative embodiments, the compound can be administered ex vivo by direct exposure to cells, tissues or organs originating from a subject.

In some embodiments, the antimalarial agent or combination of antimalarial agents, including the compounds described herein, or a pharmaceutically acceptable salt thereof, may be administered to animals, such as chickens, as an additive to their prepared feed or grain.

In some embodiments, a pharmaceutically effective amount of a compound herein, or a pharmaceutically acceptable salt thereof, may be administered to a human in need thereof by injection. In some embodiments, the injection may be subcutaneous. In other embodiments, the injection is intramuscular.

The forms in which the compound of Formula I, or a pharmaceutically acceptable salt or co-crystal thereof, may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline may also conventionally be used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

In some embodiments, the compound, or a pharmaceutically acceptable salt thereof, may be administered using a formulation comprising sesame oil, preferably pharmaceutical grade sesame oil. Components of an injectable formulation may include additional polar compounds, such as those selected from the group of monoglycerides, diglycerides, free fatty acids, plant sterols, sesamin, and sesamolin. Some injectable formulations further comprise ethanol. In some embodiments, the injectable formulation comprises from about 5 weight % to about 10 weight % ethanol. In other embodiments , the injectable formulation comprises from about 7 weight % to about 8 weight % ethanol. In other embodiments , the injectable formulation comprises from about 7.25 weight % to about 7.75 weight % ethanol. In other embodiments , the injectable formulation comprises about 7.5 weight % ethanol.

Additional components that may be used for intramuscular injections include vegetable oils, such as peanut oil, almond oil, olive oil, castor oil, and soybean oil. Also suitable are synthetic oils, such as polyethylene glycol, triglycerides of higher saturated fatty acids, monoesters of higher fatty acids, etc.

The injectable composition may also comprise one or more excipients, such as benzyl alcohol or benzoic acid compounds, including benzyl benzoate or sodium benzoate. Other useful excipients include methyl cholate, hydrophobic colloidal anhydrous silica, colloidal silicon dioxide, cholesteryl fatty acid ester like cholesteryl oleate, cholesteryl nonanoate, cholesteryl stearate, polyoxyethylen(5)sorbitan monooleate, polyoxyethylen(6) stearate, polyvalent metal salts of fatty acids e.g. aluminium stearate, fatty acid ester of carbohydrates like Rheopearl®, sorbitan fatty acid esters like sorbitan monolaurate, sorbitan sesquioleate, and sorbitan monostearate, and glycerol fatty acid ester like glycerol monostearate.

Sterile injectable solutions are prepared by incorporating a compound according to the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. In some embodiments, for parenteral administration, sterile injectable solutions are prepared containing a therapeutically effective amount, e.g., 0.1 to 1000 mg, of the compound of Formula I, or a pharmaceutically acceptable salt or co-crystal thereof. It will be understood, however, that the amount of the compound actually administered usually will be determined by a physician, in the light of the relevant circumstances, including the condition to be treated, the chosen route of administration, the actual compound administered and its relative activity, the age, weight, and response of the individual subject, the severity of the subject's symptoms, and the like.

To formulate the pharmaceutical compositions, the compound can be combined with various pharmaceutically acceptable additives, as well as a base or vehicle for dispersion of the compound. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween 80 or Miglyol 812), solubility enhancing agents (for example, cyclodextrins and derivatives thereof), stabilizers (for example, serum albumin), and reducing agents (for example, glutathione) can be included. Adjuvants, such as aluminum hydroxide (for example, Amphogel, Wyeth Laboratories, Madison, N.J.), Freund's adjuvant, MPL™ (3-O-deacylated monophosphoryl lipid A; Corixa, Hamilton, Ind.) and IL-12 (Genetics Institute, Cambridge, Mass.), among many other suitable adjuvants well known in the art, can be included in the compositions. When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The compound can be dispersed in a base or vehicle, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, microspheres and films for direct application to a mucosal surface.

The compound can be combined with the base or vehicle according to a variety of methods, and release of the compound can be by diffusion, disintegration of the vehicle, or associated formation of water channels. In some circumstances, the compound is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43:1-5, 1991), and dispersed in a biocompatible dispersing medium, which yields sustained delivery and biological activity over a protracted time.

The compositions of the disclosure can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering the compound can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

In certain embodiments, the compound can be administered in a time release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the compound and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present disclosure include, but are not limited to, polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acid-co-glycolic acid), poly(D-lactic acid-co-glycolic acid), and poly(L-lactic acid-co-glycolic acid). Other useful biodegradable or bioerodable polymers include, but are not limited to, such polymers as poly(epsilon-caprolactone), poly(epsilon-aprolactone-CO-lactic acid), poly(epsilon.-aprolactone-CO-glycolic acid), poly(beta-hydroxy butyric acid), poly(alkyl-2-cyanoacrilate), hydrogels, such as poly(hydroxyethyl methacrylate), polyamides, poly(amino acids) (for example, L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

The pharmaceutical compositions of the disclosure typically are sterile and stable under conditions of manufacture, storage and use. Sterile solutions can be prepared by incorporating the compound in the required amount in an appropriate solvent with one or a combination of ingredients enumerated herein, as required, followed by filtered sterilization. Generally, dispersions are prepared by incorporating the compound and/or other biologically active agent into a sterile vehicle that contains a basic dispersion medium and the required other ingredients from those enumerated herein. In the case of sterile powders, methods of preparation include vacuum drying and freeze-drying which yields a powder of the compound plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

In some embodiments, the method of delivering a pharmaceutically effective amount of a compound described herein, or a pharmaceutically acceptable salt thereof, may be administered to a subject in need thereof through a medical implant, particularly an implant designed to provide a continuous release, sustained release, or timed release of the active compound. In some embodiments, the implant comprises an amount of the desired compound and an ethylene vinyl acetate (EVA) copolymer, such as the copolymer designs described in U.S. Pat. No. 7,736,665 (Patel et al.), U.S. Pat. No. 8,852,623 (Patel et al.), U.S. Pat. No. 9,278,163 (Patel et al.), U.S. Pat. No. 10,111,830 (Patel et al.), and U.S. Pat. No. 10,123,971 (Patel et al.), each granted to Titan Pharmaceuticals, Inc.

In some embodiments, an implant may comprise dimensions of from 0.5 to about 7 mm in diameter. In some embodiments the devices are about 0.5 to 10 cm in length. In one embodiment, the device is from about 1 to about 3 cm in length. In one embodiment, the device is about 2 cm to about 3 cm in length. In another embodiment, the device is about 2.6 cm in length. In one embodiment, the device is about 1 to about 3 mm in diameter. In another embodiment, the device is about 2 to about 3 mm in diameter. In one embodiment, the device is about 2.4 mm in diameter. In some embodiments in which devices comprises dimensions of about 2.4 mm in total diameter and about 2.6 cm in total length, the devices each release 1 mg of pharmaceutical substance per day.

In some embodiments the implantable devices comprise from about 10% by weight to about 85% by weight a compound of Formula (I), or a pharmaceutically acceptable salt thereof, and the remainder of the implant comprises an ethylene vinyl acetate (EVA) copolymer. In some embodiments, the implant comprises about 75% active drug (a compound of Formula I, or a pharmaceutically acceptable salt thereof) and about 25% EVA. In other separate embodiments the implant comprises, respectively about 10% active drug/about 90% EVA, about 20% active drug/about 80% EVA, about 30% active drug/about 70% EVA, about 40% active drug/about 60% EVA, about 50% active drug/about 50% EVA, about 60% active drug/about 40% EVA, about 70% active drug/about 30% EVA, and about 80% active drug/about 20% EVA.

Additional embodiments comprise methods in which the active drug described herein (a compound of Formula I, or a pharmaceutically acceptable salt thereof) is administered to a subject in need thereof in a continuous release, sustained release, or timed release gel formulation, such as a hydrogel formulation. Gel carriers useful for delivering a pharmaceutically effective amount of a compound described herein, or a pharmaceutically acceptable salt thereof, include thermally responsive hydrogel carriers, including, but not limited to injectable block copolymer-based thermally responsive hydrogels; carbapol (poly-acrylic acid) gels; chitosan gels, such as chitosan thermogels; nanoparticle-containing/nanocomposite hydrogels; modified poly(ethylene glycol) gels; carrageenan gels; and water-in-sorbitan-monostearate gels. Examples of useful gel carriers include those described in Sarah Gordon's chapter Gels as Vaccine Delivery Systems at pages 203-220 in Subunit Vaccine Delivery, Springer New York 2015 (Print ISBN: 1-4939-1416-2), U.S. Pat. No. 10,272,140 (Yu et al.), U.S. Pat. No. 9,526,787 (Ko et al.), and Bobbala et al., AAPS J. 2016 January, 18(1), pp. 261-269.

Oral gel, gel-bead, or gel droplet formulations may also be used for delivering effective amounts of the compounds herein, or pharmaceutically acceptable salts thereof, to animals, such as poultry. Examples of gel formulations that may be used with the active drugs described herein include those described in U.S. Pat. No. 10,155,034 (Lee) and U.S. Pat. No. 8,858,959 (Jenkins et al.).

In accordance with the various treatment methods of the disclosure, the compound can be delivered to a subject in a manner consistent with conventional methodologies associated with management of the disorder for which treatment or prevention is sought. In accordance with the disclosure herein, a prophylactically or therapeutically effective amount of the compound and/or other biologically active agent is administered to a subject in need of such treatment for a time and under conditions sufficient to prevent, inhibit, and/or ameliorate a selected disease or condition or one or more symptom(s) thereof.

Typical subjects intended for treatment with the compositions and methods of the present disclosure include humans, as well as non-human primates and other animals. To identify subjects for prophylaxis or treatment according to the methods of the disclosure, accepted screening methods are employed to determine risk factors associated with a parasitic infection to determine the status of an existing disease or condition in a subject. These screening methods include, for example, preparation of a blood smear from an individual suspected of having malaria. The blood smear is then fixed in methanol and stained with Giemsa and examined microscopically for the presence of Plasmodium infected red blood cells. These and other routine methods allow the clinician to select patients in need of therapy using the methods and pharmaceutical compositions of the disclosure.

The administration of the compound of the disclosure can be for either prophylactic or therapeutic purpose. When provided prophylactically, the compound is provided in advance of any symptom. The prophylactic administration of the compound serves to prevent or ameliorate any subsequent disease process. When provided therapeutically, the compound is provided at (or shortly after) the onset of a symptom of disease or infection.

For prophylactic and therapeutic purposes, the compound can be administered to the subject by the oral route or in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the compound can be provided as repeated doses within a prolonged prophylaxis or treatment regimen that will yield clinically significant results to alleviate one or more symptoms or detectable conditions associated with a targeted disease or condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, avian, porcine, feline, non-human primate, and other accepted animal model subjects known in the art. Alternatively, effective dosages can be determined using in vitro models (for example, whole cell assays that monitor the effect of various drugs on parasite growth rate). Using such models, only ordinary calculations and adjustments are required to determine an appropriate concentration and dose to administer a therapeutically effective amount of the compound (for example, amounts that are effective to elicit a desired immune response or alleviate one or more symptoms of a targeted disease). In alternative embodiments, an effective amount or effective dose of the compound may simply inhibit or enhance one or more selected biological activities correlated with a disease or condition, as set forth herein, for either therapeutic or diagnostic purposes.

The actual dosage of the compound will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the compound for eliciting the desired activity or biological response in the subject. Dosage regimens can be adjusted to provide an optimum prophylactic or therapeutic response. A therapeutically effective amount is also one in which any toxic or detrimental side effects of the compound and/or other biologically active agent is outweighed in clinical terms by therapeutically beneficial effects. A non-limiting range for a therapeutically effective amount of a compound and/or other biologically active agent within the methods and formulations of the disclosure is about 0.01 mg/kg body weight to about 20 mg/kg body weight, such as about 0.05 mg/kg to about 5 mg/kg body weight, or about 0.2 mg/kg to about 2 mg/kg body weight.

Dosage can be varied by the attending clinician to maintain a desired concentration at a target site (for example, the lungs or systemic circulation). Higher or lower concentrations can be selected based on the mode of delivery, for example, trans-epidermal, rectal, oral, pulmonary, or intranasal delivery versus intravenous or subcutaneous delivery. Dosage can also be adjusted based on the release rate of the administered formulation, for example, of an intrapulmonary spray versus powder, sustained release oral versus injected particulate or transdermal delivery formulations, and so forth.

The instant disclosure also includes kits, packages and multi-container units containing the herein described pharmaceutical compositions, active ingredients, and/or means for administering the same for use in the prevention and treatment of diseases and other conditions in mammalian subjects. Kits for diagnostic use are also provided. In one embodiment, these kits include a container or formulation that contains one or more of the conjugates described herein. In one example, this component is formulated in a pharmaceutical preparation for delivery to a subject. The conjugate is optionally contained in a bulk dispensing container or unit or multi-unit dosage form. Optional dispensing means can be provided, for example a pulmonary or intranasal spray applicator. Packaging materials optionally include a label or instruction indicating for what treatment purposes and/or in what manner the pharmaceutical agent packaged therewith can be used.

### Synthesis

The present prodrugs are synthesized in one step from ELQ-300 using potassium carbonate, tetrabutylammonium iodide (TBAI), and the appropriate chloromethyl alkoxycarbonate ester in dimethyl formamide (DMF) at 60° C. The conversion is complete overnight in high yield. The alkoxycarbonate ester prodrugs have significantly lower melting points than ELQ-300, indicating a reduction in crystallinity compared to ELQ-300, i.e., ELQ-300 decomposes at ˜314° C. whereas the melting point for ELQ-331 is 103.5° C. Melting points for the chemical series of chain length-variants of ELQ-331 are provided in Table 1 along with their relative solubility limits in sesame oil, a viscous oil that is commonly used as a vehicle for intramuscular formulations.

ELQ-331, ((6-chloro-7-methoxy-2-methyl-3-(4-(4-(trifluoromethoxy)phenoxy)phenyl)quinolin-4-yl)oxy) methyl ethyl carbonate, can be prepared by methods known in the art, including the method described by Frueh et al., Alkoxycarbonate Ester Prodrugs of Preclinical Drug Candidate ELQ-300 for Prophylaxis and Treatment of Malaria, ACS Infectious Diseases, 2017, 3, pp. 728-735, and in US 2018/0362465 (Riscoe et al.). ELQ-300 referenced herein is 6-Chloro-7-methoxy-2-methyl-3-[4-[4-(trifluoromethoxy)phenoxy]phenyl]-4(1H)-quinolinone.

### Step 1

A solution of the appropriate starting alcohol (1.1 equivalents) and triethylamine (2 equivalents) in dry tetrahydrofuran (THF) was cooled to -78° C. under Argon atmosphere (see scheme below). Chloromethyl chloroformate (1 equivalent) was added dropwise and the solution was allowed to warm to room temperature overnight. The resulting chloromethyl alkyl carbonate was concentrated and used without further purification.

### Step 2

To a solution of ELQ-300 (1 equivalent) in dimethylformamide (DMF) was added tetrabutylammonium iodide (2 equivalents), potassium carbonate (2 equivalents), and the appropriate chloromethyl alkyl carbonate intermediate (2 equivalents) (FIG. 3). The solution was stirred overnight at 60° C., concentrated in vacuo, and purified by flash chromatography (silica gel, 0-80% ethyl acetate in hexane).

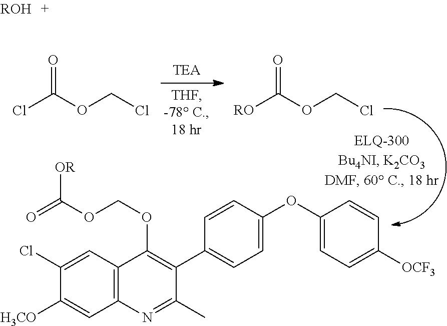

### Example Synthesis of ELQ-490, i.e., the Pentyl Side Chain Variant of ELQ-331

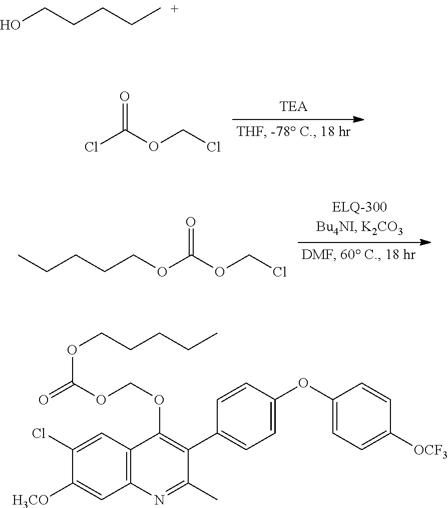

A solution of pentanol (970 mg, 11 mmol, 1.1 equivalents) and triethylamine (2.02 g, 20 mmol, 2 equivalents) in dry THF (60 mL) was cooled to −78° C. under Argon atmosphere. Chloromethyl chloroformate (1.3 g, 10 mmol, 1 equivalent) was added dropwise and the solution was allowed to warm to room temperature overnight. The resulting chloromethyl pentyl carbonate was concentrated and used without further purification.

To a solution of ELQ-300 (330 mg, 0.7 mmol, 1 equivalent) in DMF (15 mL) was added tetrabutylammonium iodide (480 mg, 1.3 mmol, 2 equivalents), potassium carbonate (180 mg, 1.3 mmol, 2 equivalents), and the chloromethyl pentyl carbonate intermediate (230 mg, 1.3 mmol, 2 equivalents). The solution was stirred overnight at 60° C., concentrated, and purified by flash chromatography, yielding an off-white waxy solid.

Analogous procedures were used to prepare each of the alkoxycarbonate chain length variants from C₁ to C₁₀ in the ELQ-331 series. In each case the final recrystallization step is from 5% ethylacetate in hexane. The chemical properties of each derivative is provided in Table 1 while analytical details may be found below.

TABLE 1

| | | | | | Sesame |
|---|---|---|---|---|---|
| | | | | Melting | Oil |
| ELQ | Notebook | | Molecular | Point | Solubility |
| Number | Number(s) | Sidechain | Weight | (° C.) | (mg/mL) |
| ELQ-487 | AK2091 | methyl | 563 | 124 | 91 |
| ELQ-331 | ELQ-331 | ethyl | 577 | 107 | 125 |
| ELQ-488 | AK2077 | n-propyl | 591 | 131 | 83 |
| ELQ-489 | AK2078 | n-butyl | 605 | 95 | 71 |
| ELQ-490 | AK2011/79 | n-pentyl | 619 | 109 | 83 |
| ELQ-491 | AK2012/80 | n-hexyl | 633 | 79 | 100 |
| ELQ-492 | AK2013/82 | n-heptyl | 647 | 63 | 71 |
| ELQ-493 | AK2014/83 | n-octyl | 661 | 54 | 100 |
| ELQ-494 | AK2015/84 | n-nonyl | 675 | 57 | 333 |
| ELQ-495 | AK2016/85 | n-decyl | 689 | 55 | ≥500 |

Generalized method for estimation of compound solubility in sesame oil: Sesame oil (10 μl) was dispensed into a glass vial containing 10 mg of the appropriate ELQ-300 prodrug. The mixture was vortexed and warmed (using a heat gun) to aid dissolution. The solubility of the compound was judged after the sesame oil mixture cooled to room temperature. If the compound was not completely dissolved, additional sesame oil was added in low-volume increments. Importantly, complete dissolution was assured by confirming by visual inspection that the compound remained in solution for at least 72 hrs at room temperature.

### NMR Data for ELQ-300 alkoxycarbonate Ester Prodrugs

### ((6-chloro-7-methoxy-2-methyl-3-(4-(4-(trifluoromethoxy)phenoxy)phenyl)quinolin-4-yl)oxy)methyl pentyl carbonate

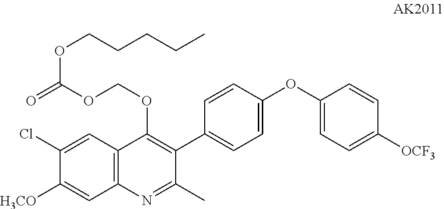 [SMILES: CCCCCOC(=O)OCOc1c(-c2ccc(Oc3ccc(OC(F)(F)F)cc3)cc2)c(C)nc2cc(OC)c(Cl)cc12]

¹H-NMR (400 MHz; DMSO-d₆): δ 7.72; (s, 1H, ArH), 7.57; (s, 1H, ArH), 7.47-7.41; (m, 4H, ArH), 7.23-7.18; (m, 4H, ArH), 5.37; (s, 2H, CH₂), 4.04; (s, 3H, CH₃), 2.45; (s, 3H, CH₃), 1.27-1.13; (m, 8H, CH₂), 0.81; (t, J=8, 3H CH₃).

### ((6-chloro-7-methoxy-2-methyl-3-(4-(4-(trifluoromethoxy)phenoxy)phenyl)quinolin-4-yl)oxy)methyl hexyl carbonate

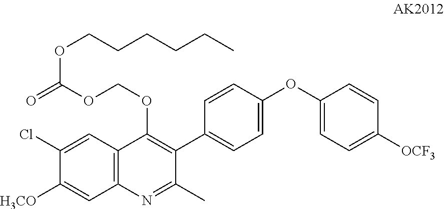 [SMILES: CCCCCCOC(=O)OCOc1c(-c2ccc(Oc3ccc(OC(F)(F)F)cc3)cc2)c(C)nc2cc(OC)c(Cl)cc12]

¹H-NMR (400 MHz; DMSO-d₆): δ 7.99; (s, 1H, ArH), 7.57; (s, 1H, ArH), 7.45-7.41; (m, 4H, ArH), 7.31-7.16; (m, 4H, ArH), 5.35; (s, 2H, CH₂), 4.03; (s, 3H, CH₃), 2.44; (s, 3H, CH₃), 1.45-1.17; (m, 10H, CH₂), 0.82; (t, J=8, 3H CH₃).

### ((6-chloro-7-methoxy-2-methyl-3-(4-(4-(trifluoromethoxy)phenoxy)phenyl)quinolin-4-yl)oxy)methyl heptyl carbonate

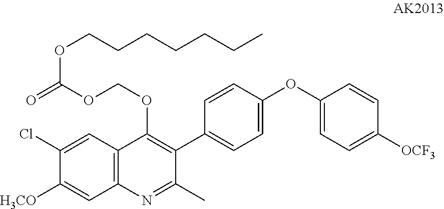 [SMILES: CCCCCCCOC(=O)OCOc1c(-c2ccc(Oc3ccc(OC(F)(F)F)cc3)cc2)c(C)nc2cc(OC)c(Cl)cc12]

¹H-NMR (400 MHz; DMSO-d₆): δ 7.89; (s, 1H, ArH), 7.57; (s, 1H, ArH), 7.46-7.43; (m, 4H, ArH), 7.23-7.18; (m, 4H, ArH), 5.35; (s, 2H, CH₂), 4.03; (s, 3H, CH₃), 2.44; (s, 3H, CH₃), 1.46-1.17; (m, 12H, CH₂), 0.84; (t, J=8, 3H CH₃).

### ((6-chloro-7-methoxy-2-methyl-3-(4-(4-(trifluoromethoxy)phenoxy)phenyl)quinolin-4-yl)oxy)methyl octyl carbonate

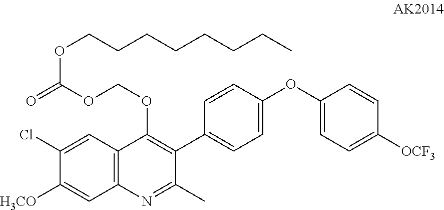 [SMILES: CCCCCCCCOC(=O)OCOc1c(-c2ccc(Oc3ccc(OC(F)(F)F)cc3)cc2)c(C)nc2cc(OC)c(Cl)cc12]

¹H-NMR (400 MHz; DMSO-d₆): δ 7.95; (s, 1H, ArH), 7.53; (s, 1H, ArH), 7.44-7.41; (m, 4H, ArH), 7.29-7.07; (m, 4H, ArH), 5.34; (s, 2H, CH₂), 4.03; (s, 3H, CH₃), 2.44; (s, 3H, CH₃), 1.48-1.07; (m, 16H, CH₂), 0.86; (t, J=8, 3H CH₃).

### ((6-chloro-7-methoxy-2-methyl-3-(4-(4-(trifluoromethoxy)phenoxy)phenyl)quinolin-4-yl)oxy)methyl decyl carbonate

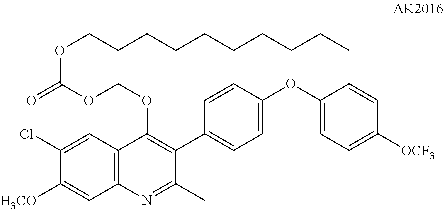 [SMILES: CCCCCCCCCCOC(=O)OCOc1c(-c2ccc(Oc3ccc(OC(F)(F)F)cc3)cc2)c(C)nc2cc(OC)c(Cl)cc12]

¹H-NMR (400 MHz; DMSO-d₆): δ 7.98; (s, 1H, ArH), 7.56; (s, 1H, ArH), 7.45-7.42; (m, 4H, ArH), 7.23-7.18; (m, 4H, ArH), 5.35; (s, 2H, CH₂), 4.03; (s, 3H, CH₃), 2.44; (s, 3H, CH₃), 1.48-1.18; (m, 18H, CH₂), 0.85; (t, J=8, 3H CH₃).

### Pharmacokinetics of Intramuscular (im) Injections of ELQ-331

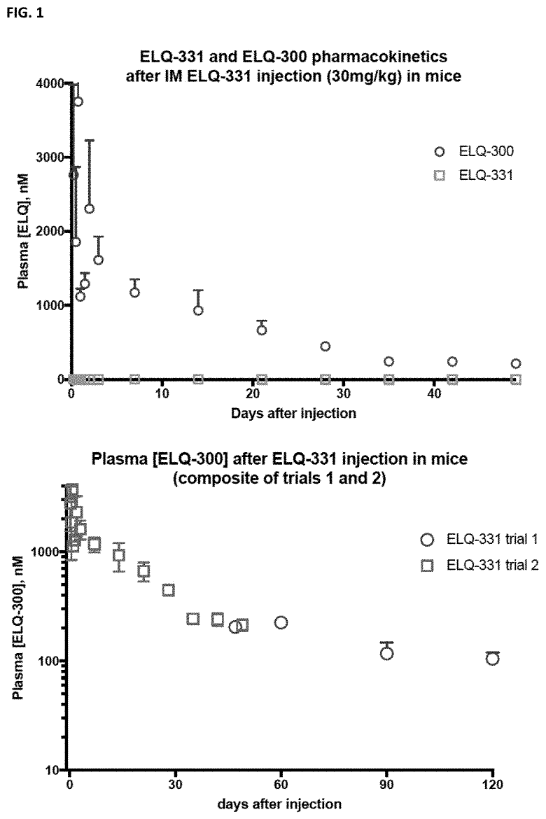

We dissolved ELQ-331 in sesame oil and administered (30 mg/kg) in a single 50 μm injection into the caudal thigh of mice in two separate trials. Trial 1 served as a pilot study to monitor ELQ-300 blood levels for months following a single im injection. In Trial 2 we adjusted the LC-MS/MS chromatographic protocol to capture prodrug [ELQ-331] as well as [ELQ-300] with early time-points to cover the critical burst phase, a phenomenon known as “dose dumping”. As shown in FIG. 1, Trial 1 plasma levels of ELQ-300 above 83 nM (i.e., the minimum effective concentration cited by Chatterjee in their model system at CALIBR, presented at the 2017 ASTMH meeting in Baltimore, Md.), were maintained for at least 120 days. In Trial 2, we monitored plasma [ELQ-331] and [ELQ-300] starting 6 hours after im injection. To avoid excessive blood draws, 6 different groups of 4 mice, all treated at the same time, were used in a rotating succession, one group for each time point. FIG. 1 depicts Plasma [ELQ-300], nM, following im injection in mice of 30 mg/kg ELQ-331 (prodrug) in sesame oil. The first graph shows values for the early phase following im injection during which ELQ-300 levels fluctuate somewhat before beginning a slow and steady decline for 120 days. At all time-points the [ELQ-300] concentration exceeds the Minimum Fully Effective Concentration (MEC₁₀₀) of ≈60 nM. All data points are the average values±SEM.

As shown in FIG. 1, maximum [ELQ-300] approached 4 μM in the “burst phase” between 6 and 18 hours after injection with initial variability likely due to differences in the depth and location of depot placement within the muscle. Variability soon diminished, yielding a consistent curve suggesting a multi-compartment PK profile. Importantly, at every time point, the ratio of ELQ-300/ELQ-331 is very large; i.e., nearly all drug present is in the form of ELQ-300 which suggests that combined esterase activity is capable of quickly converting ELQ-331 to ELQ-300. Also shown in FIG. 5 is a composite plot of plasma [ELQ-300] from both trials with remarkable consistency in the results. ELQ-331 outperformed the CALIBR ELQ-300 prodrug (mCAR246) profiled at the 2017 ASTMH meeting by a significant margin, i.e., Day 28 bloodstream [ELQ-300] levels for mCAR246=181±59 (30 mg/kg, unknown formulation) vs. 446±22 nM (avg.±SEM) for prodrug ELQ-331 in (30 mg/kg, sesame oil). These impressive PK results from ELQ-331 in a simple, stable and inexpensive sesame oil vehicle are amplified by the findings below that this dosing conferred complete protection from sporozoite-induced infection 4½ months after treatment.

### Sesame Oil im Sustained-Release Formulation of ELQ-331: Protection against P. yoelii Sporozoite-Induced Infection in Mice

Animals receiving an im injection of 30 mg/kg ELQ-331 in sesame oil were challenged by inoculation with 10,000 P. yoelii luciferase expressing parasites (PyXNL non-lethal strain, luc⁺) at two different time points, 48 days and 137 days post im injection (Note that separate groups of mice were used for these two challenges). For details of our experimental methods please refer to the work proposed section as well as published papers by Qigui Li (17) and Kami Kim (18). Sporozoites were obtained by dissection of infected mosquitoes, A. stephensi, and purification over glass wool with assistance from Dr. Brandon Wilder (formerly Sack), who recently joined the OHSU faculty (see letter of collaboration). Non-invasive whole animal

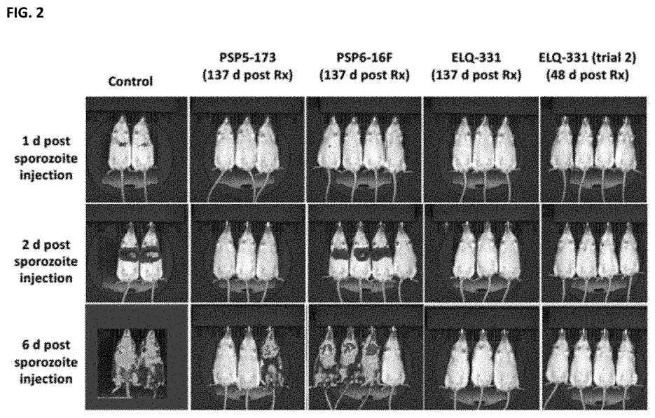

Bioluminescence imaging (BLI) was used to evaluate all study animals for the presence of liver stage infection using an IVIS Spectrum imager. Briefly, on the day of analysis mice received a single ip injection with a luciferin solution roughly 10 minutes before imaging. Each animal was placed on the IVIS platform with gaseous isoflurane serving as anesthetic. Notice in FIG. 2 that vehicle only control animals exhibited a robust BLI signal in the region corresponding to the liver of Py luc⁺-infected mice. Most impressively, there was no detectable bioluminescent signal in mice that had received an im injection with 30 mg/kg ELQ-331 both 48 (Trial 2, above) and 137 (Trial 1, above) days prior to sporozoite challenge, a finding that is consistent with inhibition of liver stage parasite development by plasma levels of ELQ-300 that remained at 105±15 nM on Day 120. Of interest, we also challenged animals that had received an im injection (dissolved in sesame oil) of two similar ELQ-300 prodrugs, PSPS-173 and PSP6-16F.

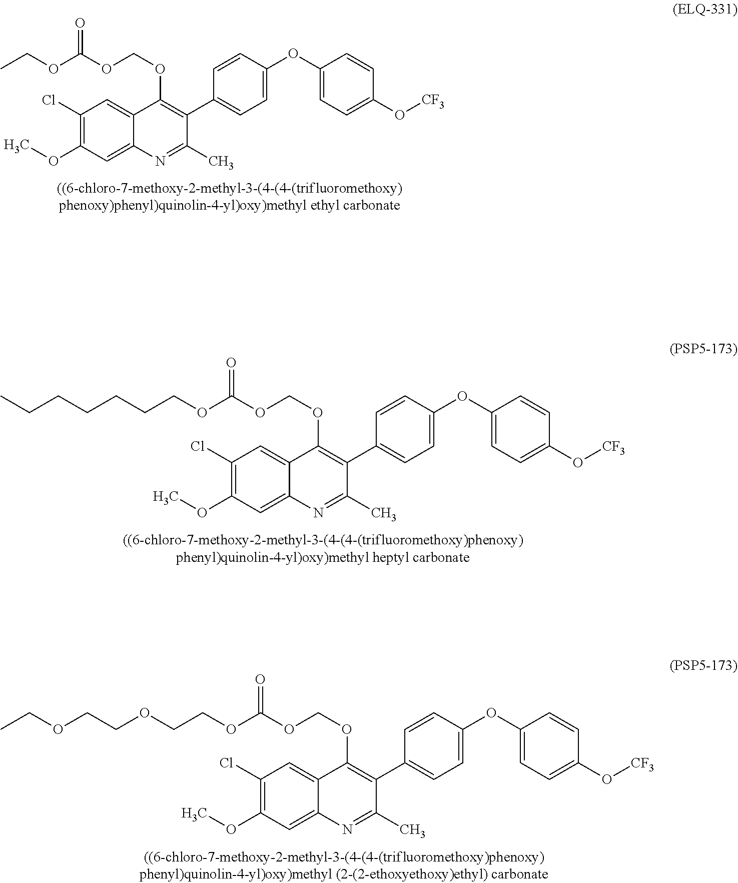 [SMILES: CCOC(=O)OCOc1c(-c2ccc(Oc3ccc(OC(F)(F)F)cc3)cc2)c(C)nc2cc(OC)c(Cl)cc12 ; CCCCCCCOC(=O)OCOc1c(-c2ccc(Oc3ccc(OC(F)(F)F)cc3)cc2)c(C)nc2cc(OC)c(Cl)cc12 ; CCOCCOCCOC(=O)OCOc1c(-c2ccc(Oc3ccc(OC(F)(F)F)cc3)cc2)c(C)nc2cc(OC)c(Cl)cc12]

Day 120 plasma levels of [ELQ-300] for these two drugs were 72±13 nM (PSP5-173, i.e., the C7-straight-chain carbonate ester) and 65.5±40 (i.e., the alkoxycarbonate ester with transcutol in the ester position). One of three mice (note that group size varied from 3 to 4 mice in the pilot) in the PSP5-173 group had no visible liver signal but developed a blood stage infection, and 3 of 4 mice in the PSP6-16F group exhibited a detectable liver signal, albeit weaker than that of controls, and developed blood stage infection. These efficacy results matched exactly the rank order of day 120 plasma [ELQ-300] between and within treatment groups. Day 120 plasma [ELQ-300] were <40 nM in 3 of 4 mice treated with PSP6-16F, <60 nM in 1 of 3 treated with PSP5-173, but were all >80 nM in those treated with ELQ-331. Taken together our results show that a simple and inexpensive sesame oil formulation of ELQ-331 provides impressive long-term protection against sporozoite induced infections in mice. While the site of ELQ-300 activity as a chemo-preventive is intrahepatic, and thus blood concentration is merely a proxy, a conservative starting estimate of the Minimum Fully Effective Concentration of ELQ-300 (MEC₁₀₀) in our P. yoelii model based on extrapolation of available data is ≈60 nM (i.e., 28.6 ng/ml).

Bioluminescent imaging (FIG. 2) was used to analyze the protective effect of im ELQ-300 prodrugs on Plasmodium yoelii liver stages. Mice were infected by inoculation with 10,000 Plasmodium yoelii GFP/luciferase expressing parasites (PyXNL non-lethal strain, luc*). As described in the narrative infected mice received sporozoite injections 48 days and 137 days following im injection with selected ELQ-300 prodrugs dissolved in sesame oil. After sporozoite challenge, mice underwent imaging for detection of bioluminescent signal after 24 and 48 hours and then again on Day 6 post-inoculation. Control mice show bioluminescent signal in the region above the liver of anesthetized mice after 24 hours which becomes more intense by 48 hours after which parasites emerge into the bloodstream as evidenced by widespread signal intensity across the entire animal by 6-days post inoculation. Animals treated with ELQ-331 at 30 mg/kg were fully protected against sporozoite challenge even after 48 and 137 days following a single im injection. Mice treated with either PSP5-173 or PSP6-16F, with demonstrably lower bloodstream ELQ-300 levels, were only partially protected from sporozoite challenge at day 137 post inoculation.

Implications of preliminary data. This evidence clearly shows sustained release and rapid conversion of ELQ-331 to ELQ-300 after im injection, confirming that gut esterase activity occurring after oral dosing is not required for effective conversion to ELQ-300. Furthermore, these results point to the very real potential for formulation optimization to diminish the burst phase to decrease host exposures to unnecessarily high concentrations of ELQ-300 (i.e., even though 4 μM is well below the NOAEL level determined by the recent non-GLP 7-day repeat dose toxicity/toxicokinetic study in rats of ELQ-331/ELQ-300 described above). Lastly, the ability to achieve more than 4 months of protection in mice with a single im injection of a dose far below any proposed toxicity threshold is strong evidence that development of an ELQ-300 prodrug im formulation for sustained-delivery and long-term protection against malaria in humans is entirely feasible.

What is claimed:

1. A compound of Formula (I): 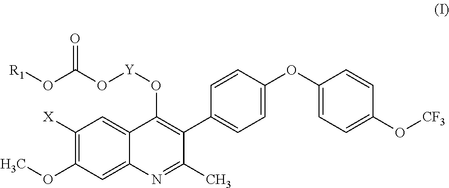
wherein:
X is selected from the group of F and CI;
Y is selected from the group of —CH₂—, —CH₂—CH₂—, and —CH₂(CH₃)—;
R₁ is selected from the group of:
a) —CH3;
b) n-propyl;
c) C₄-C₂₀ linear or branched alkyl;
d) —(C₄-C₂₀ branched or linear alkylene)-C₃-C₆ cycloalkyl;
e) —(C₄-C₂₀ branched or linear alkylene)-C₃-C₆ heterocyclyl;
f) —(C₄-C₂₀ branched or linear alkylene)-C₃-C₆ cycloalkene;
g) —(C₄-C₂₀ branched or linear alkylene)-phenyl;
h) —(C₄-C₂₀ branched or linear alkylene)-naphthyl;
i) C₃-C₆ cycloalkyl;
j) C₃-C₆ heterocyclyl;
k) C₃-C₆ cycloalkene;
l) phenyl;
m) naphthyl;
n) C2-C20 linear or branched alkene;
o) —(C₂-C₂₀ linear or branched alkenylene)-C₃-C₆ cycloalkyl;
p) —(C₂-C₂₀ linear or branched alkenylene)-C₃-C₆ heterocycle;
q) —(C₂-C₂₀ linear or branched alkenylene)-C₃-C₆ cycloalkene;
r) —(C₄-C₂₀ branched or linear alkenylene)-phenyl;
s) —(C₄-C₂₀ branched or linear alkenylene)-naphthyl;
t) C₂-C₂₀ linear or branched alkyne;
u) —(C₂-C₂₀ linear or branched alkynylene)-C₃-C₆ cycloalkyl;
v) —(C₂-C₂₀ linear or branched alkynylene)-C₃-C₆ heterocycle;
w) —(C₂-C₂₀ linear or branched alkynylene)-C₃-C₆ cycloalkene;
x) —(C₄-C₂₀ branched or linear alkynylene)-phenyl;
y) —(C₄-C₂₀ branched or linear alkynylene)-naphthyl;
and
z) —CH₂—CH₂O—)₁₋₅—R₂;
wherein the branched alkyl chain of c), the branched alkylene chain of groups d), e), f), and g), the branched alkenylene groups of o), p), q), r), and s), and the branched alkynylene groups of u), v), w), x), and y) are substituted with 1, 2, 3, 4, or 5 substituents selected from the group of C₁-C₆ alkyl, C₂-C₆ alkenyl, C₂-C₆ alkynyl, C₃-C₆ cycloalkyl, —(C₁-C₃ alkylene)-C₃-C₆ cycloalkyl, 3-6 membered heterocyclyl groups, and —(C₁-C₃ alkylene)-3-6 membered heterocyclyl;
and
R₂ is selected from the group of methyl and ethyl;
or a pharmaceutically acceptable salt thereof.

2. The compound of claim 1, or a pharmaceutically acceptable salt thereof, wherein Y is —CH₂—.

3. The compound of claim 1, or a pharmaceutically acceptable salt thereof, wherein R₁ is C₄-C₁₀ branched or linear alkylene.

4. The compound of claim 1, or a pharmaceutically acceptable salt thereof, wherein R₁ is R₁ is C₁₀-C₂₀ branched or linear alkylene.

5. The compound of claim 1, or a pharmaceutically acceptable salt thereof, wherein R₁ is —(CH₂—CH₂—O—)₁₋₅—R₂ and R₂ is methyl or ethyl.

6. The compound of claim 1, wherein the compound is selected from the group of: 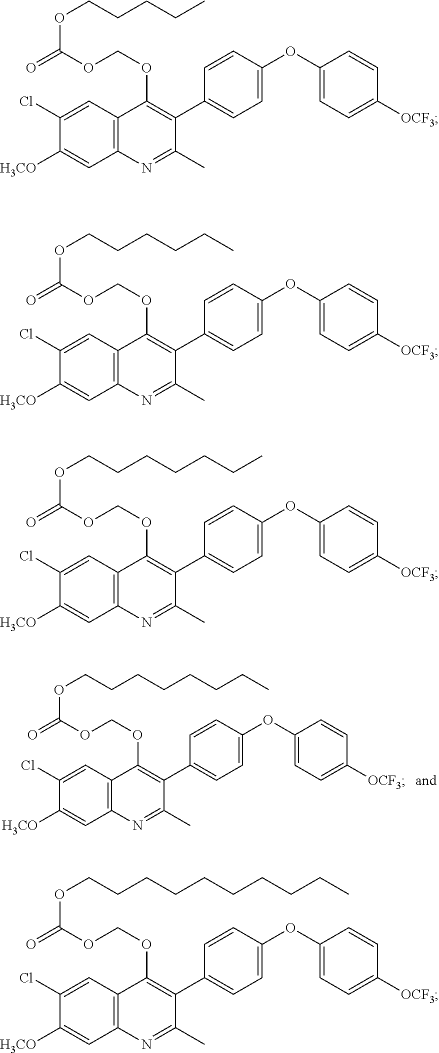 [SMILES: CCCCCOC(=O)OCOc1c(-c2ccc(Oc3ccc(OC(F)(F)F)cc3)cc2)c(C)nc2cc(OC)c(Cl)cc12 ; CCCCCCOC(=O)OCOc1c(-c2ccc(Oc3ccc(OC(F)(F)F)cc3)cc2)c(C)nc2cc(OC)c(Cl)cc12 ; CCCCCCCOC(=O)OCOc1c(-c2ccc(Oc3ccc(OC(F)(F)F)cc3)cc2)c(C)nc2cc(OC)c(Cl)cc12 ; CCCCCCCCOC(=O)OCOc1c(-c2ccc(Oc3ccc(OC(F)(F)F)cc3)cc2)c(C)nc2cc(OC)c(Cl)cc12 ; "and" ; CCCCCCCCCCOC(=O)OCOc1c(-c2ccc(Oc3ccc(OC(F)(F)F)cc3)cc2)c(C)nc2cc(OC)c(Cl)cc12]
or a pharmaceutically acceptable salt thereof.

7. A pharmaceutical composition comprising a pharmaceutically effective amount of a compound of

1. , or a pharmaceutically acceptable salt thereof, and a pharmaceutically acceptable carrier or excipient.

8. The pharmaceutical composition of claim 7, wherein the compound of claim 1 is selected from the group of: 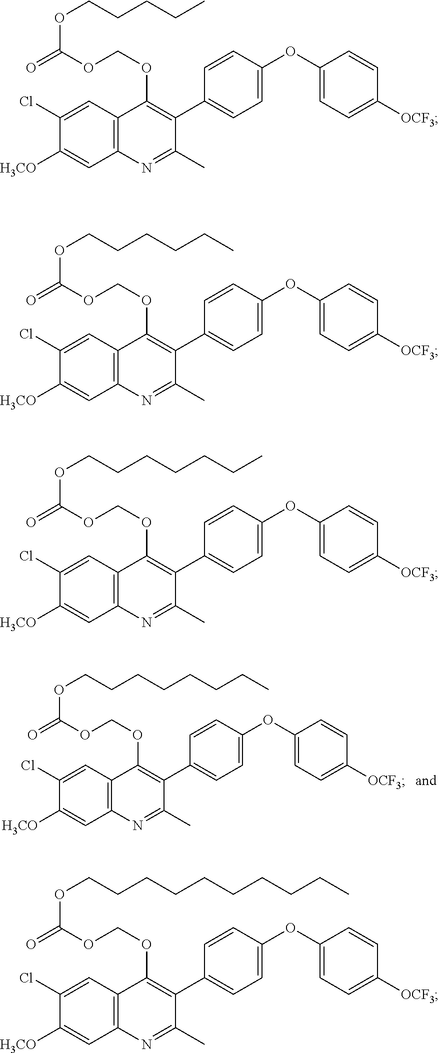 [SMILES: CCCCCOC(=O)OCOc1c(-c2ccc(Oc3ccc(OC(F)(F)F)cc3)cc2)c(C)nc2cc(OC)c(Cl)cc12 ; CCCCCCOC(=O)OCOc1c(-c2ccc(Oc3ccc(OC(F)(F)F)cc3)cc2)c(C)nc2cc(OC)c(Cl)cc12 ; CCCCCCCOC(=O)OCOc1c(-c2ccc(Oc3ccc(OC(F)(F)F)cc3)cc2)c(C)nc2cc(OC)c(Cl)cc12 ; CCCCCCCCOC(=O)OCOc1c(-c2ccc(Oc3ccc(OC(F)(F)F)cc3)cc2)c(C)nc2cc(OC)c(Cl)cc12 ; "and" ; CCCCCCCCCCOC(=O)OCOc1c(-c2ccc(Oc3ccc(OC(F)(F)F)cc3)cc2)c(C)nc2cc(OC)c(Cl)cc12]
or a pharmaceutically acceptable salt thereof.

9. A method for treating malaria or toxoplasmosis in a human in need thereof, the method comprising

1. tering to the human a pharmaceutically effective amount of a compound of claim 1, or a pharmaceutically acceptable salt thereof.

10. The method for treating malaria or toxoplasmosis in a human in need thereof of claim 7, wherein the compound of claim 1 is selected from the group of: 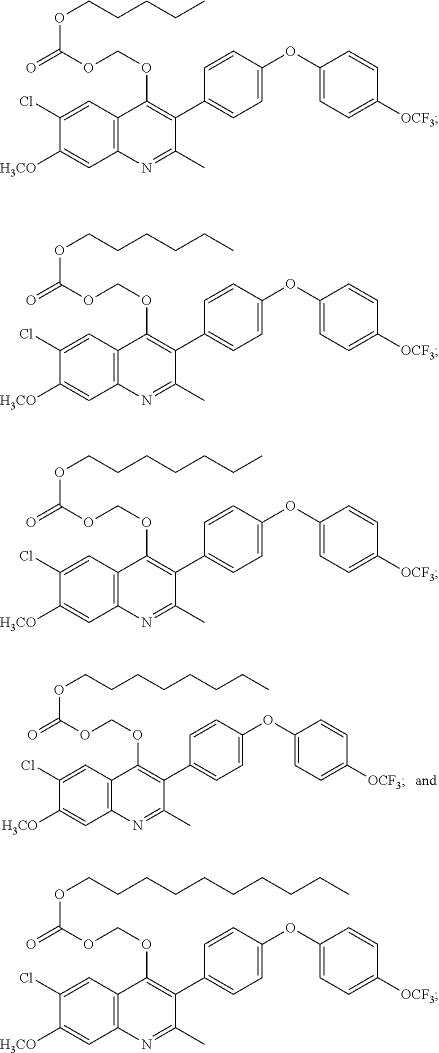 [SMILES: CCCCCOC(=O)OCOc1c(-c2ccc(Oc3ccc(OC(F)(F)F)cc3)cc2)c(C)nc2cc(OC)c(Cl)cc12 ; CCCCCCOC(=O)OCOc1c(-c2ccc(Oc3ccc(OC(F)(F)F)cc3)cc2)c(C)nc2cc(OC)c(Cl)cc12 ; CCCCCCCOC(=O)OCOc1c(-c2ccc(Oc3ccc(OC(F)(F)F)cc3)cc2)c(C)nc2cc(OC)c(Cl)cc12 ; CCCCCCCCOC(=O)OCOc1c(-c2ccc(Oc3ccc(OC(F)(F)F)cc3)cc2)c(C)nc2cc(OC)c(Cl)cc12 ; "and" ; CCCCCCCCCCOC(=O)OCOc1c(-c2ccc(Oc3ccc(OC(F)(F)F)cc3)cc2)c(C)nc2cc(OC)c(Cl)cc12]
or a pharmaceutically acceptable salt thereof.

11. The method of claim 10, wherein the malaria is multidrug-resistant malaria.

12. The method of claim 11, wherein the malaria is chloroquine-resistant malaria.

13. The method of claim 10, wherein the pharmaceutically effective amount of the compound, or a pharmaceutically acceptable salt thereof, is administered to the human in need thereof by intramuscular injection.
